(19) United States
(12) Patent Application Publication
(10) Pub. No.: US 2006/0143718 A1
(43) Pub. Date: Jun. 29, 2006

(54) Transgenic animals for monitoring water quality

(76) Inventor: Daniel W. Nebert, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 11/286,613

(22) Filed: Nov. 23, 2005

Related U.S. Application Data
(63) Continuation of application No. 09/863,528, filed on May 22, 2001.
(60) Provisional application No. 60/206,196, filed on May 22, 2000.

(51) Int. Cl. A01K 67/027

(52) U.S. Cl. 800/3; 800/20

(57) ABSTRACT

The present invention provides methods and systems that uses transgenic zebrafish with an easily assessable reporter gene under the control of pollutant-inducible DNA response elements. Transgenic zebrafish, carrying pollution-inducible response elements, are placed in the water to be tested, and the contaminants become bioconcentrated (generally 1,000- to 40,000-fold, relative to the water) in the tissues of the fish thereby activating specific response elements, which up-regulate the LUC reporter gene. Fish are then removed from the test water and placed immediately in a luminometer cuvette and incubated with luciferin. Luciferin is rapidly taken up into the tissues of the fish, oxidized by luciferase, and light is produced. The luminescence is proportional to the environmental concentration of the pollutant (to which the fish had been exposed), which drives the expression of the LUC gene by means of the various DNA motifs. The luminescence is quantitated in the luminometer. In each response element-containing construct, a specific class of polluting chemicals, allowing for differential identification of pollutants in a complex mixture activates the expression of the LUC gene. This assay does not require killing the fish and allows for repeated analysis of the same site with the same fish. The sensitivity of the system can be manipulated by varying the sequence of the response element.

### RELATED APPLICATIONS

This application is a continuation of U.S. patent application Ser. No. 09/863,528, filed May 22, 2001, which claims priority of U.S. Provisional Patent Appl. Ser. No. 60/206,196, filed May 22, 2000, which are both specifically incorporated by reference herein without disclaimer.

This invention was made in part with Government support under Grant No. R01-ES07058, awarded by the National Institute of Environmental Health Sciences. The Government may have certain rights in this invention.

### FIELD OF THE INVENTION

The present invention relates to using transgenic animals for monitoring water quality. In particular, the present invention provides methods and materials for transgenic lines in which DNA motifs that respond to select environmental pollutants are capable of activating a reporter gene that can be easily assayed.

### BACKGROUND OF THE INVENTION

Exposure to numerous man-made and natural environmental agents poses a significant threat to human health. For many of these dangerous toxic agents, aquatic environments serve as the major route of distribution, and their sediments represent the ultimate sink. Human exposure to many aquatic pollutants occurs primarily through the ingestion of contaminated fish and/or shellfish. Fish accumulate environmental contaminants by absorption across the gill epithelium, and primarily, by bioconcentration in the food chain. It has been demonstrated that this bioconcentration can be in excess of 40,000 times for Hg and 100,000 times for TCDD. Humans, unfortunately, are at the end of the food chain.

In order to protect human health, regulatory agencies have set limits on the concentrations levels and kinds of pollutants allowed entering bodies of water. These water-quality criteria are established on the basis of correlations between the concentration of a pollutant in a body of water and the accumulation of that pollutant in fish; ultimately, these data are extrapolated to risk assessment methodologies in humans. There is no mathematical formula in which the concentration of a particular contaminant, measured at its source, can be correlated to a concentration of that contaminant in fish.

In monitoring the quality of the aquatic environment, a major approach involves the quantitation of water, sediment, or tissue residue levels by analytical chemical methods, which are generally expensive, labor-intensive, and slow. This process usually includes the acquisition of a sample in the field, transport back to the analytical facility, sample processing, data collection, and, finally, data analysis. This is the more straightforward of the methods used-but also the more expensive, requiring extensive technical expertise in the analysis of pesticide, inorganic, non-pesticide organic, physical, and radiological parameters.

Wild-caught fish are often used as a biomonitor to indicate the potential for human exposure to polycyclic hydrocarbon, oxidant and metal contaminants. This method is proposed to circumvent problems related to correlating effluent concentrations at the source to concentrations in fish tissues. The evaluation of fish tissues for the presence of dangerous foreign chemical(s) is also quite expensive and labor-intensive, and of limited utility, because the bioavailability of a particular chemical(s) in the body of water is often unknown.

In fish, the most common assays require the collection of specimens and preparation of the appropriate tissue and/or biochemical samples (i.e., liver homogenate, DNA, etc.). Specific assays have traditionally involved the analysis of DNA damage, factors that regulate redox potential in the cell (glutathione, ascorbic acid and tocopherol), or quantitation of the activity of enzymatic defenses such as superoxide dismutase, catalase and glutathione peroxidase. Changes in the expression of pollutant-inducible genes have also been used to indicate the exposure to a wide variety of contaminants. Such analyses require specialized equipment found in laboratories that use the latest molecular biological tools, specialized training in the use of such tools, and great care in sample handling to limit denaturing relevant mRNA and proteins.

Although environmental pollutants are known to act upon several fish enzyme systems, there are inherent limitations in the interpretation of such data because a number of physiological, genetic, and metabolic factors have an impact on these multifunctional enzyme complexes. Individual variability is likely to be striking when measurements from several fish are taken. Moreover, the fish tissues require great care in handling so as to try to limit denaturation and/or proteolysis.

It has long been established that environmental contaminants are bioconcentrated in fish and other aquatic organisms. The degree of bioconcentration will vary depending upon the species, type of contaminant (due to solubility in water), the organism's capacity for metabolism and excretion, and chemical properties of the water (e.g. concentration of ionic and organic material affecting solubility). However, related chemical contaminants under standard conditions will be bioconcentrated to a similar degree for most species of fish. Contaminant levels in wild fish are often 1,000 to 100,000 times higher than levels in their environment. For example, mercury levels can be more than 40,000 times higher in fish muscle tissue as compared with that in the surrounding water. TCDD has been reported to become bioconcentrated 100,000-fold in fish. This means that 10⁻¹⁷ M TCDD in the water or sediments would be bioconcentrated in fish to about 10⁻¹² M (0.32 parts per trillion) levels and might activate the transcription of at least some of the dioxin-inducible genes, of which there are several dozen genes. It is this process of gene induction, combined in an organism that bioconcentrates polluting chemicals, that is used in the present invention.

### SUMMARY OF THE INVENTION

The present invention provides methods and systems that uses transgenic zebrafish with an easily assessable reporter gene under the control of pollutant-inducible DNA response elements. Transgenic zebrafish, carrying pollution-inducible response elements, are placed in the water to be tested, and the contaminants become bioconcentrated (generally 1,000- to 40,000-fold, relative to the water) in the tissues of the fish thereby activating specific response elements, which up-regulate the LUC or GFP reporter genes. Fish are then removed from the test water and placed immediately in a luminometer cuvette and incubated with luciferin. Luciferin is rapidly taken up into the tissues of the fish, oxidized by luciferase, and light is produced. The luminescence is proportional to the environmental concentration of the pollutant (to which the fish had been exposed), which drives the expression of the LUC or GFP gene by means of the various DNA motifs. The luminescence is quantitated in the luminometer. In each response element-containing construct, the expression of the LUC or GFP gene is activated by a specific class of polluting chemicals, allowing for differential identification of pollutants in a complex mixture. This assay does not require killing the fish and allows for repeated analysis of the same site with the same fish. The sensitivity of the system can be manipulated by varying the sequence of the response element.

This zebrafish model system provides sensitive, economical and practical biological monitors for specific common aquatic pollutants, and should be able to differentiate between chemical classes within a complex mixture. The only equipment required to detect luciferase activity is a luminometer. In this living system, the only reagent needed is luciferin.

There are several advantages of this model system in the detection of aquatic pollutants. First, data analysis is much faster. Environmental agents generally become bioconcentrated in fish in a matter of minutes. Luciferase readings from 20 zebrafish, which might indicate (for example) a specific increase in Hg concentrations, can be achieved in less than 30 min including the time required for luciferin uptake. Traditional analytical chemical methods take days from the time of sampling to the determination of pollutant values. Second, data acquisition is significantly cheaper and, thus, allows for the sampling of more sites. Traditional analytical chemical equipment is expensive. Shipping samples to a central analytical facility might reduce the cost per sample but greatly increases the time required for data acquisition and analysis. Luciferase readings from these zebrafish can be analyzed in the back of a truck, or in a boat, with a luminometer and a laptop computer connected to a regular automobile (or boat) battery. Third, in vivo bioaccumulation in fish is a much better indicator of potential exposure via consumption of contaminated fish than is the analysis of water and/or sediment samples.

Fish are the direct source of most pollutant exposure, and, as described above, fish are able to bioconcentrate pollutants in their environment. If water-borne pollution, rather than fish consumption, is the concern for estimating human exposure, then analyzing fish for biological effects will also give us a better understanding of the bioavailability of aquatic pollutants.

### BRIEF DESCRIPTION OF THE DRAWINGS

This invention, as defined in the claims, can be better understood with reference to the following drawings. The drawings are not necessarily to scale, emphasis instead being placed upon clearly illustrating principles of the present invention.

FIG. 1 is a schematic diagram of the transgenic zebrafish model system as a sentinel for monitoring of aquatic pollution. Transgenic zebrafish, carrying pollution-inducible response elements, are placed in the water to be tested, and the contaminants (*) are bioconcentrated in the tissues of the fish thereby activating any specific response element (RE) which then up-regulates the LUC or GFP gene. The higher the concentration of pollutant, the greater the luminescence in this assay. In each response element-containing construct, a specific class of polluting chemicals, allowing for the differential identification of pollutants in a complex mixture activates the expression of the LUC gene. The sensitivity of the system can be manipulated by varying the copy number, and the nucleotide sequence, of the response element.

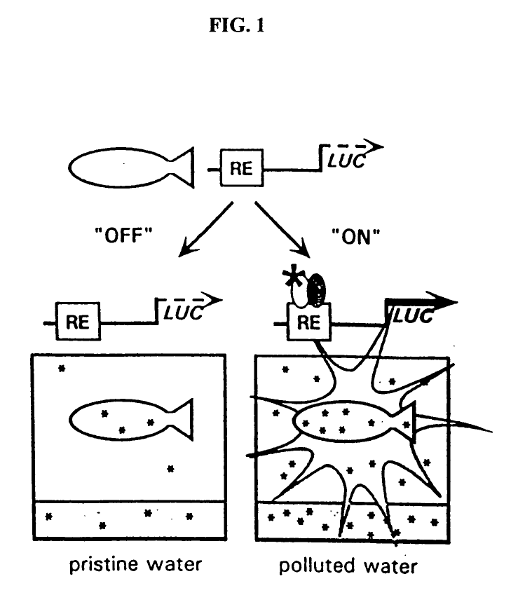

FIG. 2 is a comparison of inducible promoters in zebrafish ZEM2S cells. Reporter constructs included DNA sequences from the 5′ regulatory regions of mammalian and trout genes, cloned into the pGL3-Basic firefly luciferase (LUC) reporter construct. Promoter/enhancer sequences (from left to right) were derived from: mouse Cyp1a1 (−1646 to +57); mouse AhRDtk [−1100 to −896 of mouse Cyp1a1 containing four AHREs, fused to the herpes simplex virus type I thymidine kinase (tk) minimal promoter (−79 to +53) from which the SP1-binding site was removed]; rainbow trout CYP1A3 (−1987 to +78); human CYP1A1 (−1604 to +88); mouse EPREmt1 [single EPRE from the mouse Gsta1 enhancer region (−722 to −682) fused to the minimal mouse Mt1 promoter]; mouse Nqo1 (from the Mlu I restriction site at approximately −3000 to +109); human NQO1 (−1539 to +115); mouse MREd₅mt1 [concatamer of five MREd′ sequences from the mouse Mt1 enhancer fused to the minimal mouse Mt1 promoter (−42 to +60)]; and the trout MT-B promoter/enhancer sequences (−137 to +8). Following transient transfection, we determined that maximal LUC activity was achieved in AHRE reporter constructs by 10 nM TCDD, in EPRE constructs by 10 μM tBHQ, and in MRE constructs by 30 μM CdCl₂. The data represent the means of duplicate determinations from at least six independent transfections and brackets denote standard errors of the mean.

### DETAILED DESCRIPTION OF THE INVENTION

The present invention provides for a method and animal system for a biological monitor of aquatic environmental pollution. Specifically, this invention provides transgenic zebrafish in which DNA response elements that respond to select environmental pollutants are able to activate an easily assessable reporter gene.

A transgenic animal is an animal having cells that contain a transgene, wherein the transgene was introduced into the animal or an ancestor of the animal at a prenatal, e.g., an embryonic, stage. A transgene is a DNA which is integrated into the genome of a cell from which a transgenic animal develops and which remains in the genome of the mature animal, thereby directing the expression of an encoded gene product in one or more cell types or tissues of the transgenic animal.

A transgenic animal can be created, for example, by introducing a nucleic acid encoding the fusion protein (generally operatively linked to appropriate regulatory elements) into the male pronuclei of a fertilized oocyte, e.g., by microinjection, and allowing the oocyte to develop in a female foster animal. Methods for generating transgenic animals have become conventional in the art and are described, for example, in U.S. Pat. Nos. 4,736,866 and 4,870,009, incorporated herein by reference. A transgenic founder animal can be used to breed additional animals carrying the transgene.

“Operably linked” refers to a juxtaposition wherein the components so described are in a relationship permitting them to function in their intended manner. For instance, a promoter is operably linked to a coding sequence if the promoter affects its transcription or expression. The term “promoter” is a region of DNA involved in binding RNA polymerase to initiate transcription.

A transgenic cell or animal contains one or more transgenes within its genome. A transgene is a DNA sequence integrated at a locus of a genome, wherein the transgenic DNA sequence is not otherwise normally found at that locus in that genome. Transgenes may be made up of heterologous or homologous DNA sequences.

When the term DNA is used herein, it should be understood that for the number of purposes where DNA can be substituted with RNA, the term DNA should be read to include RNA embodiments which will be apparent to one skilled in the art.

The term “homologous” is used here to illustrate the degree of identity between the amino acid sequence of a given polypeptide. The amino acid sequence may be deduced from a DNA sequence, e.g. obtained by hybridization as defined above, or may be obtained by conventional amino acid sequencing methods. The degree of homology is preferably determined on the amino acid sequence of a mature polypeptide, i.e. without taking any leader sequence into consideration. It is preferred that the degree of homology is generally at least about 85%, preferably at least about 90%, more preferably at least about 95% and most preferably at least about 98% with the known amino acid sequence.

In the present context, the term “gene” is used to indicate a DNA sequence which is involved in producing a polypeptide chain and which includes regions preceding and following the coding region (5′ -upstream and 3′ -downstream sequences) as well as intervening sequences (“introns”) which are placed between individual coding segments (“exons”) or in the 5′-upstream or 3′-downstream region. The 5′-upstream region comprises one or more regulatory sequences that control the expression of the gene, typically a promoter. The 3′-downstream region comprises sequences that are involved in termination of transcription of the gene and the 3′ untranslated region.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

In general, the 5′ expression regulation sequence includes the transcribed portion of the endogenous gene upstream from the translation initiation sequence (the 5′ untranslated region or 5′ UTR) and those flanking sequences upstream therefrom which comprise a functional promoter.

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eucaryotic promoters will often, but not always, contain “TATA” boxes and “CAT” boxes. Procaryotic promoters contain Shine-Dalgarno sequences in addition to the −10 and −35 consensus sequences.

DNA “control sequences” refer collectively to promoter sequences, ribosome binding sites, polyadenylation signals, transcription termination sequences, upstream regulatory domains, enhancers, and the like, which collectively provide for the transcription and translation of a coding sequence in a host cell.

A control sequence “directs the transcription” of a coding sequence in a cell when RNA polymerase will bind the promoter sequence and transcribe the coding sequence into mRNA, which is then translated into the polypeptide encoded by the coding sequence.

In addition to a promoter, the transgene may contain one or more enhancer and/or other sequences that facilitate expression of the endogenous gene and as a consequence facilitate the expression of the structural DNA sequence operably linked to the regulation sequences. Although the use of both 5′ and 3′ regulation sequences are preferred, in some cases, 3′ regulation sequences are not used. It is to be understood that the recombinant polypeptide encoded by the transgene may comprise either genomic DNA or a double stranded DNA derived from cDNA. The transgenes of the invention generally also comprises one or more intron sequences that interrupt the transcribed region of the transgene.

The DNA sequences of the invention explained herein may comprise natural as well as synthetic DNA sequences, the natural sequence typically being derived directly from cDNA or genomic DNA, normally of mammalian origin, e.g. as described below. A synthetic sequence may be prepared by conventional methods for synthetically preparing DNA molecules. DNA sequences may be mixed cDNA and genomic, mixed cDNA and synthetic and mixed genomic and synthetic origin. Also RNA sequences may be used.

The transgenic animals of the invention are produced by introducing a “transgene” into an embryonal target cell of the animal of choice. In one aspect of the invention, a transgene is a DNA sequence that is capable of producing a desirable phenotype when contained in the genome of cells of a transgenic non-human animal. The incorporation of the expression system into the germline of the animal may be performed using any suitable technique.

Gene transfer systems known in the art may be useful in the practice of the methods of the present invention. These include viral and nonviral transfer methods. A number of viruses have been used as gene transfer vectors, including papovaviruses, e.g., SV40, adenovirus, vaccinia virus, adeno-associated virus, herpesviruses including HSV and EBV, and retroviruses of avian, murine and human origin. Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer. Viral-mediated gene transfer can be combined with direct in vivo gene transfer using liposome delivery.

A cell has been “transformed” by exogenous DNA when such exogenous DNA has been introduced inside the cell membrane. Exogenous DNA may or may not be integrated (covalently linked) to chromosomal DNA making up the genome of the cell. In some cell systems, the exogenous DNA may be maintained on an episomal element, such as a plasmid. With respect to eucaryotic cells, a transformed cell is one in which the exogenous DNA has become integrated into the chromosome so that it is inherited by daughter cells through chromosome replication. This stability is demonstrated by the ability of the eucaryotic cell to establish cell lines or clones comprised of a population of daughter cell containing the exogenous DNA.

The present invention utilizes the firefly luciferase (luc) or green fluorescent protein (GFP) as the reporter gene in zebrafish because the assay is extremely sensitive, rapid, easy to perform and relatively inexpensive.

The zebrafish model system has many advantages that have been exploited in recent years for investigations of developmental genetics and cancer genetics. Many of the same characteristics make the zebrafish an attractive experimental system for studying the biological and toxic effects evoked by xenobiotics in fish. Finally, use of the LUC or GFP reporter gene in a transgenic zebrafish gives an assay using the living fish, monitored by a luminometer, as a convenient nonmammalian alternative model for assessing the levels of aquatic pollution.

The transgenic zebrafish (Danio rerio) function as sensitive, economical, and practical biological monitors for specific common aquatic pollutants. DNA response elements that respond to selected classes of environmental pollutants regulate the induction of luciferase, an easily assessable reporter gene. The response elements are chosen as ones known to respond to classes of environmental pollutants that are found at significant levels in the aquatic environment and, as a result, pose a threat to human health through exposure in drinking water and by consumption of contaminated fish and/or shellfish.

Zebrafish oocytes and fertilized eggs are generally transparent and easy to use for microinjection. They hatch in 2-3 days and have a relatively short generation time of 3-4 months. Well-characterized transcription control elements from viruses and mammals are able to direct protein expression in fish cells. More recently, promoter elements isolated from fish species have been analyzed for their capacity to direct protein synthesis in fish cells and transgenic animals.

Many pollutants, like mercury, are increasing in the aquatic environment and there is a need to monitor an ever-increasing number of water bodies. Transgenic fish biomonitoring system for detecting pollutants allows for the efficient, low-cost monitoring of many additional sites and for the majority of the time, effort, and dollars to be expended on the sites that need it the most. The advantages of a transgenic fish biomonitoring system for detecting increases in pollutant levels are several. First, data analysis would be much faster. Luciferase readings from 20 zebrafish that would indicate a specific increase in Hg concentrations can be acquired in less than 30 minutes. Traditional analytical chemical methods take days from the time of sampling to the determination of pollutant values.

Second, data acquisition would be significantly cheaper and thus allow for the sampling of more sites. Traditional analytical chemical equipment is expensive. Shipping samples to a central analytical facility reduces the cost per sample but greatly increases the time required for data acquisition and analysis. Luciferase readings from these fish can be analyzed in the back of a truck with a luminometer connected to its battery and a laptop computer.

Third, in vivo bioaccumulation in fish is a much better indicator of potential exposure via consumption of contaminated fish than is the analysis of water samples. Fish are the direct source of most pollutant exposure and bioconcentrate pollutants in their environment. Mercury, as in our example, can be more than 40,000 times higher in fish muscle tissue compared to the water body. If water-borne pollutants are the concern for human exposure, not fish consumption, analyzing fish for biological effects will give a better understanding of the bioavailability of pollutants.

Luminescence is a phenomenon in which energy is specifically channeled to a molecule to produce an excited state. Return to a lower energy state is accompanied by release of a photon. Luminescence includes fluorescence, phosphorescence, chemiluminescence and bioluminescence.

Bioluminescence is the process by which living organisms emit light that is detectable. Where the luminescence is bioluminescence, creation of the excited state derives from an enzyme-catalyzed reaction. The color of the emitted light is characteristic of the excited molecule, and is independent from its source of excitation and temperature.

An essential condition for bioluminescence is the use of molecular oxygen, either bound or free in the presence of a luciferase. Luciferases, are oxygenases that act on a substrate, luciferin, in the presence of molecular oxygen and transform the substrate to an excited state. Upon return to a lower energy level, energy is released in the form of light.

Bioluminescent molecules are distinguished from fluorescent molecules in that they do not require the input of radiative energy to emit light. Rather, bioluminescent molecules utilize chemical energy, such as ATP, to produce light. As used herein, luminescence refers to the detectable EM radiation, generally, UV, IR or visible EM radiation that is produced when the excited product of an exergic chemical process reverts to its ground state with the emission of light.

Several types of bioluminescent molecules are known. They include the luciferase family and the aequorin family. Luciferase is a stable, monomeric protein that does not require posttranslational modification for enzymatic activity and is not found in vertebrate systems, eliminates endogenous background and “false positive” measurements.

Members of the luciferase family have been identified in a variety of prokaryotic and eucaryotic organisms. Luciferase and other enzymes involved in the prokaryotic luminescent (lux) systems, as well as the corresponding lux genes, have been isolated from marine bacteria in the Vibrio and Photobacterium genera and from terrestrial bacteria in the Xenorhabdus genus.

The luciferase system (luc) has been found in the firefly Photinus pyralis. The firefly contains in its abdomen the enzyme protein, luciferase (LUC), and the enzyme's substrate, luciferin. Its glow is produced when the firefly somehow allows the luciferin to come into contact with the enzyme, in the presence of an energy source called ATP.

Luciferase” or “luc”, unless stated otherwise, includes prokaryotic and eucaryotic luciferases, as well as variants possessing varied or altered optical properties, such as luciferases that luminesce at wavelengths in the red range. As used herein, the term “lux” refers to prokaryotic genes associated with luciferase and photon emission. As used herein, the term “luc” refers to eucaryotic genes associated with luciferase and photon emission.

Bioluminescent proteins that are present in a variety of marine invertebrates, such as the green and blue fluorescent proteins, particularly the green fluorescent protein (GFP) of Aequorea victoria, may also be used. “Green fluorescent protein” or “GFP” constitute a class of chromoproteins found only among certain bioluminescent coelenterates.

These accessory proteins are fluorescent and function as the ultimate bioluminescence emitter in these organisms by accepting energy from enzyme-bound, excited-state oxyluciferin. The best-characterized GFPs are those isolated from the jellyfish species Aequorea, particularly Aequorea victoria (A. victoria) and Aequorea forskalea.

The present invention utilizes the firefly luciferase (luc) gene inserted into zebrafish as a reporter gene, preferably driven by response elements of the CYP1A1, NMO1 and MT genes. Luciferase is a stable, monomeric protein that does not require posttranslational processing for enzymatic activity and is not found in normal vertebrate systems, limiting endogenous background and “false positive” measurements. The luciferase reaction proceeds as shown in the following Equation I:

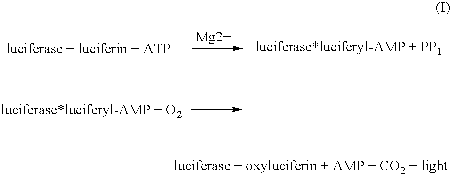

The only equipment required to detect luciferase activity is a luminometer. In a living system, the only reagent needed is luciferin. Therefore, the present invention provides a living animal, a zebrafish, comprising exogenous genetic material comprising a DNA molecule having one or more regulatory elements from a gene operatively linked to a DNA sequence encoding one or more reporter elements. The “a regulatory element” from a gene is the DNA sequence that is necessary for the transcription of the gene.

The regulatory element in the present invention is a pollutant-inducible DNA response element. Preferably, pollutant-inducible DNA response element is a modular enhancer unit or response element selected from the group consisting of the metal response element (MRE), the aromatic hydrocarbon response element (AHRE), the estrogen response element (ERE), the electrophile response element (EPRE), and the retinoic acid response elements (RARE, RXRE).

The response element controls the expression of the reporter element by controlling the transcription of the reporter. The reporter element is a bioluminescent luciferase system (luc or lux) or GFP system.

The above-described zebrafish are useful to monitor water quality. As such, the present invention provides for a method for using transgenic zebrafish with an easily assessable reporter gene under the control of pollutant-inducible DNA response elements. Transgenic zebrafish, carrying pollution-inducible response elements, are placed in the water to be tested, and the contaminants become bioconcentrated (generally 1,000- to 40,000-fold, relative to the water) in the tissues of the fish thereby activating specific response elements, which up-regulate the LUC or GFP reporter genes. Generally, the fish are then removed from the test water and placed immediately in a luminometer cuvette and incubated with luciferin. Luciferin is rapidly taken up into the tissues of the fish, oxidized by luciferase, and light is produced. The luminescence is proportional to the environmental concentration of the pollutant (to which the fish had been exposed), which drives the expression of the LUC or GFP gene by means of the various DNA motifs. The luminescence is quantitated in the luminometer. In each response element-containing construct, the expression of the LUC or GFP gene is activated by a specific class of polluting chemicals, allowing for differential identification of pollutants in a complex mixture.

In another embodiment, the invention provides a method of measuring contaminants in water comprising:
a. introducing into a transgenic zebrafish organism a DNA construct having the sequence of the regulatory response element gene operatively linked to a DNA molecule encoding a reporter gene such that a regulatory element of the gene controls expression of the reporter gene;
b. exposing the transgenic zebrafish to a water sample to be tested for a time sufficient to allow contaminants within the water sample to become bioconcentrated within the zebrafish;
c. exposing the transgenic zebrafish to conditions permitting expression of the reporter gene; and
d. detecting the expression of the reporter gene.

In another embodiment, the invention provides a method of measuring contaminants in water comprising:
a. introducing into a transgenic zebrafish organism a DNA construct having the sequence of at least one regulatory response element gene operatively linked to a DNA molecule encoding at least one reporter gene such that a regulatory element of the gene controls expression of the reporter gene;
b. exposing the transgenic zebrafish to a water sample to be tested for a time sufficient to allow contaminants become bioconcentrated within the zebrafish
c. exposing the transgenic zebrafish to conditions permitting expression of the reporter gene; and
d. detecting the expression of the reporter gene; and
e. quantitating the detected expression by correlating to known standards and thereby detecting the quantity of contaminants in the water sample.

In another embodiment, the invention provides a method of measuring contaminants in water comprising:
a. introducing into a transgenic zebrafish organism a DNA construct having the sequence of at least one regulatory response element gene operatively linked to a DNA molecule encoding at least one reporter gene such that a regulatory element of the gene controls expression of the reporter gene;
b. exposing the transgenic zebrafish to a water sample to be tested for a time sufficient to allow contaminants become bioconcentrated within the zebrafish
c. exposing the transgenic zebrafish to conditions permitting expression of the reporter gene; and
d. detecting the expression of the reporter gene;
e. quantitating the detected expression by correlating to known standards and thereby detecting the quantity of contaminants in the water sample;
f. wherein the regulatory response element is a promoter.

Preferably, the response element is a metal response element (MRE), the aromatic hydrocarbon response element (AHRE), the estrogen response element (ERE), the electrophile response element (EPRE), and the retinoic acid response elements (RARE, RXRE). In another embodiment, the reference standard is an aquatic source containing a known contaminant concentration. In another embodiment, the transgene is made up of multiple copies of the response element. In yet another embodiment, the transgene contains more than one type of response element. In yet another embodiment, the transgene contains more than two types of response element. In yet another embodiment, the transgene contains two or more copies each of more than one type of response element. In yet another embodiment, the transgene contains additional promoters or enhancers. In yet another embodiment, the transgene contains response elements from

In another embodiment, the response element is from a gene selected from the group consisting of CYP1A, CYP1B, CYP1A1CYP2D6, CYP3A, CYP3A4, MT, MT1, MT2, MTF-1, ACE1, NMO1, AMT1, AHR, ARNT, AHR1, AHR2, ARNT1, ARNT2, AHRE1, AHRE2, and AHRE5.

Generally, the reporter element is a bioluminescent system. Preferably, the bioluminescent system is a luciferase or GFP system. More preferably, the bioluminescent system is a luciferase system. Most preferably, the bioluminescent system is a eucaryotic luciferase system. In another embodiment, the conditions permitting expression of the reporter gene include a sufficient amount of enzyme substrate. Generally, the substrate is luciferin. Preferably, the detecting of the expression of the reporter gene is by using a luminometer.

In another embodiment, the transgenic zebrafish is exposed to a water sample to be tested continually wherein the zebrafish is removed from the water sample repeatedly at selected intervals exposed to conditions permitting expression of the reporter gene and detected for reporter gene expression wherein such repeated exposures and detecting of expression is effective to track a time course of contaminant levels.

Generally, the contaminant to be detected is selected from the group consisting of polyaromatic hydrocarbons, electrophilic oxidants heavy metals, endocrines, and retinoids. Preferably, the contaminant to be detected is selected from the group consisting of 2,3,7,8-tetrachlorodibenzo-p-dioxin, dioxin, polychlorinated biphenyls, quinones, mercury, copper, nickel, cadmium, zinc, estrogens, retinoic acid and 9-cis-retinoic acid.

Generally, the transgenic zebrafish are exposed to a water sample to be tested for a time sufficient to allow contaminants become bioconcentrated within the zebrafish. The exposure time is generally at least one minute. Preferably at least 2 minutes, more preferably at least one hour, more preferably at least 12 hours, more preferably at least 24 hours, more preferably at least one week, and more preferably at least two weeks. When the transgenic zebrafish is to remain exposed to the sample water for a longer duration in order to take multiple readings and create a time plot of contaminant levels, the total exposure time is generally at least at least 24 hours, more preferably is a time period chosen to be at least one week, at least two weeks, at least four weeks, at least eight weeks, at least 12 weeks, at least 24 weeks and at least 52 weeks.

Generally, the contaminants become bioconcentrated in the transgenic zebrafish, when placed in the water to be tested. This is expressed as the BioConcentration Factor (“BCF”) and is defined as the concentration in the organism/concentration in the water sample. BCF will vary greatly dependent upon the species of the fish, the type of contaminant, and the chemical properties of the water. Generally, such BCF will be at least 100, preferably at least 500, and more preferably at least 1,000. Such BCF can be more than 10,000, and in some cases more than 40,000. The BCF of lindane will generally be at least 1000. The BCF for dioxin will generally be at least 1000, often at least 10,000, and even at least 30,000. The BCF for mercury contaminants will generally be at least 500, often at least 950, at least 1500, at least 2500, and at least 5000.

Some of the enhancer regions (DNA motifs) that been characterized include the metal response element (MRE), the aromatic hydrocarbon response element (AHRE), the estrogen response element (ERE), the electrophile response element (EPRE), and two retinoic acid response elements (RARE, RXRE). Heavy metals such as cadmium, zinc or mercury turn on particular genes via the MRE. Dioxin, polychlorinated biphenyls (PCBs), and benzpyrene generated in combustion processes turn on some genes via the AHRE. Environmental and natural estrogens turn on specific genes via the ERE. Oxidants such as bleaching agents and hydrogen peroxide turn on distinct genes via the EPRE. Certain retinoids turn on certain genes via the RARE and RXRE.

Inducible response elements consist of a core consensus sequence, which usually is influenced by its flanking sequences and/or nearby multiple response elements (i.e. cooperativity) in causing maximal induction. The present invention uses six response elements that recognize specific important chemical classes. Aromatic hydrocarbon response elements (AHREs) respond to a wide variety of polycyclic hydrocarbons and halogenated planar molecules such as 2,3,7,8-tetrachlorodibenzo-p-dioxin (TCDD; dioxin) and polychlorinated biphenyls (PCBs) as well as polychlorinated dibenzo-p-dioxin (PCDD), polychlorinated dibenzofuran (PCDF), and polychlorinated di-aromatic hydrocarbon (PCDH), a kind of polyaromatic hydrocarbon (PAH). Quinones and a wide variety of other potent electrophilic oxidants activate electrophile response elements (EPREs). Metal response elements (MREs) respond to heavy metals such as mercury, copper, nickel, cadmium and zinc. Estrogen response elements (EREs) are upregulated by estrogens and other environmentally important endocrine disruptors. Retinoic acid response elements (RAREs) and retinoid X receptor response elements (RXREs) respond to 9-cis-retinoic acid and other retinoids.

Some of the DNA motifs plus their core consensus sequences and basic properties that are preferred for use in the present invention are summarized in TABLE 1 and described briefly below.

TABLE 1

Some DNA motifs that respond to environmental pollutants. Several properties of the
pollution-inducible response elements are listed. Extended flanking sequences, which
may be necessary for maximal response, are highly variable and not shown. As indi-
cated, some genes can be induced by several response elements due to the complexity
of their 5′ flanking sequences or the oxidative properties of the inducing pollutant.
Within each consensus sequence N = A, T, G, or C; R = A or G; W = A or T.

| | Consensus | | | |
|---|---|---|---|---|
| Response | sequence | Activating | Transcription | Normal genes |
| element | 5′-3′ | agents | factors | up-regulated |
| AHRE | TWGCGTG | Dibenzo-p-dioxins, | AH receptor + | Cytochromes P450 1 |
| | | Dibenzofurans, | ARNT heterodimer | (CYP1A, 1B), |
| | | Planar poly- | | Quinone |
| | | chlorinated | | oxidoreductase, |
| | | biphenyls and | | Glutathione |
| | | polycyclic aromatic | | transferase, UDP |
| | | hydrocarbons | | glucuronosyl- |
| | | | | transferases |
| EPRE | RTGACNNNGC | Planar aromatic | NF-E2-related | Heme oxygenase, |
| | | hydrocarbons, | factor 1 (?), | Glutamate-cysteine |
| | | Potent electrophiles | NF-E2-related | ligase, Quinone |
| | | (heavy metals, | factor 2 (?), | oxidoreductase, |
| | | arsenicals, diphenols, | Small Maf (?), | Glutathione |
| | | quinones, azo dyes) | ARE-BP (?) | transferase, UDP |
| | | | | glucuronosyl- |
| | | | | transferase |
| MRE | TGCRCNCGG | Heavy metals | MTF-1 | Metallothioneins, |
| | | | | Glutamate-cysteine |
| | | | | ligase |
| ERE | GGTCANNNTGACC | Estrogen, | Estrogen | Estrogen-responsive |
| | | Pharmaceuticals, | receptor | finger protein, |
| | | Pesticides, | homodimer | Vitellogenin, |
| | | Chlorinated | | Glucose-6- |
| | | aromatic | | phosphatase, |
| | | hydrocarbons, | | Lactoferrin |
| | | Phytoestrogens | | |
| RARE | RGGTCA | Retinoic acid and | Retinoic acid receptor | Hoxal, |
| | (N₀₋₈) | other retinoids | homodimers, | Retinoic acid receptor, |
| | RGGTCA | natural and | heterodimers with | Cellular retinoic acid |
| | | pharmaceutical | Retinoid X receptor | binding protein II, |
| | | | | Fetoprotein |
| RXRE | GGGGTCAAAGGTCA | Retinoic acid and | Retinoid X receptor | Apolipoprotein A1 |
| | GGGGTCATGGGGTC | other retinoids | homodimers | |
| | A | natural and | | |
| | | pharmaceutical | | |

Aromatic hydrocarbon response element (AHRE). Ligands for the Ah receptor (AHR) activate the AHRE and many adverse biological effects including immunosuppression, teratogenesis, tumor promotion, endocrine disruption, and cardiovascular disease. Upon binding ligand, the AHR translocates to the nucleus and binds to AHRE motifs located in the promoter region of the mammalian CYP1A1 and probably more than a dozen other genes. Halogenated and nonhalogenated polycyclic hydrocarbons (e.g. polychlorinated biphenyls, TCDD, benzo[a]pyrene) are ligands for the AHR and, thus, activate genes via AHREs. An example of this system is U.S. Pat. Nos. 5,854,010 and 5,378,822, incorporated by reference.

Electrophile response element (EPRE). Also called “antioxidant response element” (ARE), the EPRE is activated following treatment with potent oxidants and electrophiles, leading to the induction of numerous stress-inducible genes. Electrophilic compounds and metabolites that activate EPREs also react with nucleophilic centers on macromolecules and are involved in mutagenesis, carcinogenesis and aging. Inducing agents include not only reactive hydrogen peroxide, phenols and quinones but also metabolites of phase I metabolism such as oxygenated benzo[a]pyrene or naphthoflavone. EPRE sequences have been found upstream of phase II drug-metabolizing genes and other genes that respond to oxidative stress.

Metal response element (MRE). MREs were first identified upstream of the mouse metallothionein (Mt1, Mt2) genes. Heavy metal cations that induce via the MRE include cadmium, zinc, mercury, cobalt and nickel. Several heavy metals are potent electrophiles, thus activating the EPRE as well as the MRE, leading to mutagenesis and carcinogenesis. Induction of genes via MREs occurs upon exposure to heavy metals such as cadmium, silver, copper, cobalt, mercury, and nickel; zinc and heavy metal toxicity has been demonstrated in virtually every organ system.

Estrogen response element (ERE). The estrogen receptor (“ER”) binds a number of estrogenic compounds and forms a transcription complex with the ERE as a homodimer. Environmental and dietary “endocrine disruptors” bind (to varying degrees) to the ER and are purported to disrupt normal cellular signaling and lead to reproductive tissue abnormalities and/or cancer. Several environmental and pharmaceutical chemicals exhibit varying degrees of estrogenicity including diethylstilbestrol, tamoxifen, dietary phytoestrogens, phthalate plasticizers, insecticides (e.g. p,p′-DDT, p,p′-DDE, dieldrin, methoxychlor, toxaphene, endosulfan), and 4-nonylphenol, bis-phenol-A and kepone.

Retinoic acid and retinoid X response elements (RAREs, RXREs). Both retinoic acid receptors (RARs) and retinoid X receptors (RXRs) bind with high affinity to 9-cis-retinoic acid but show striking differences in their affinity for other retinoids. Many retinoic acid analogues have been developed as therapeutic and chemopreventive agents and bind preferentially to specific RAR and/or RXR isoforms activating RAREs and RXREs. The popular insecticide methoprene has been found to be a potent RXR agonist. An imbalance in the normal levels of retinoic acid (vitamin A) and/or its derivatives can cause striking deformities in limbs and other organs during embryonic development or regeneration. Environmental retinoids have been implicated in frog deformities in the Great Lakes Area where a powerful teratogen appears to exist in groundwater and well water.

The zebrafish is an efficient vertebrate model system because of its relatively short reproductive cycle, the large number of progeny that can be produced, and the relatively small space needed to maintain large numbers of offspring at low cost. Zebrafish embryos are also transparent and accessible throughout development, which allows for easy microinjection and other manipulations. Moreover, the zebrafish is becoming a powerful system for genetic analysis with the development of a high-density genome map and intentions of the Zebrafish Genome Project to completely sequence this (comparatively small) genome within the next several years.

Relatively simple and reliable methods for the production of transgenic zebrafish have also been developed. Gene transfer into embryos has improved with the use of retroviral vectors and transposons, and the use of border elements has stabilized the expression of transgenes in subsequent generations.

Zebrafish embryos are essentially transparent and, hence, make excellent model systems for the introduction of luminous and/or fluorescent markers. It was reported that LUC activity can be detected within the deep tissues of adult mice. Therefore, we felt there should be no problem detecting LUC activity within the tissues of an adult zebrafish. The advancement of successfully expressing the jellyfish green fluorescent protein (GFP) reporter gene has also allowed for the rapid development of this probe in the zebrafish. In another embodiment, “gene swapping” methods can be used, i.e. swapping a heterotypic lox-flanked gene for gfpzeo in zebrafish embryos.

Because it is preferable to assay luminescence or fluorescence in the living intact fish, it is preferable to use zebrafish lines lacking pigmentation. Initial studies with a mutant albino line revealed this line would be difficult due to chronic poor breeding. Alternatively, the golden, long-fin zebrafish (gol/lof) zebrafish line works well because the very long fins are an excellent source of tissue for genotyping and because it has reduced amounts of body pigmentation.

Generally, for the insertion of plasmids into the zebrafish embryo, electroporation or microinjection may be used although the latter tends to be more efficient. Alternatively, transgenic animals can be made using constructs containing the locus control region (LCR) of the mouse Mt1 gene, in order to create an artificial locus. Since, it is often difficult to maintain transgenes through many subsequent generations, insulating border elements, such as the Mt1-LCR, are typically used to stabilize the expression of transgenes in zebrafish for several generations.

In monitoring water quality, various modes of contaminant exposure cages, flow-through tanks, and sediment exposure can be used as known in the art. Generally, the fish will be held in aluminum cages anchored to cement blocks submersed within specific bodies of water.

The following references, to the extent that they provide exemplary procedural or other details supplementary to those set forth herein, are specifically incorporated herein by reference: U.S. Pat. Nos. 4,800,159, 4,883,750, 4,965,188, 5,176,995, 5,441,884, 5,737,018, 6,110,693, 6,117,639, 6,133,027, 6,217,847, and 6,232,107.

### EXAMPLES

Fₒ transgenic zebrafish express transgenes into adulthood. Embryos were microinjected with supercoiled plasmid at the 1- or 2-cell stage, and visualized or assayed 24 h later. The rate at which embryos survived microinjection and expressed the transgene is shown in TABLE 2. The EF1-GFPZ-MTLCR construct gave the best embryo survival rate, and also produced a very high number of embryonic cells expressing GFPzeo. High levels of expression in these zebrafish have been maintained for more than 180 days, and the transgene has been successfully transmitted into the F₁ and, sometimes the F₂, generation following which it is lost. Other laboratories have had the same difficulties in sustaining transgene expression beyond the F₂ generation in zebrafish, for reasons not known but possibly due to an efficient genome surveillance system in this species. Another possible explanation might be related to gene silencing in mammals, plants, and Drosophila which has been observed when multiple transgene copies are incorporated into a single site.

TABLE 2
Generation of transgenic zebrafish with a variety of constructs.
The following constructs were microinjected into 1- or 2-cell embryos,
and transgene expression was determined visually 24 h later. CMV
= human cytomegalovirus promoter. EF1 = Xenopus elongation
factor promoter. gfpzeo = fusion between the GFP gene and
the Zeocin-resistance gene. Gal = β-galactosidase. MTLCR
= locus control region of the mouse Mt1 gene. PGL3 = basal
construct containing the LUC gene.

| | Fish | Survival | Transgene positive |
|---|---|---|---|
| Construct | injected | (%) | (%) |
| CMV-gfpzeo- | 356 | 26 | 58 |
| MTLCR | | | |
| EF1-gfpzeo- | 534 | 69 | 58 |
| MTLCR | | | |
| EF1-βGal | 118 | 58 | 35 |
| pGL3-control | 56 | 34 | 68 |
| AHRDtkluc3 | 144 | 63 | 81 |

Following initial characterization of two zebrafish cell lines, we determined that the ZEM2S line derived from an embryonic stem cell culture grew better and responded to inducers better than the ZFL line. We then examined whole-cell and nuclear extracts of ZEM2S cells, using electrophoretic mobility shift analysis, for their capacity to bind AHRE, EPRE or MRE motifs; we concluded that ZEM2S cells indeed appear to contain all the factors necessary to specifically bind to these response elements within well-defined limits of ligand concentrations, salt requirements, and temperature.

For transient transfection of the pGL3-control plasmid (SV40 promoter and enhancer, driving the LUC gene) into ZEM2S cells, we compared the calcium phosphate method with Lipofectin (Life Technologies, Grand Island, N.Y.), Lipofectamine (Life Technologies), Lipofectamine Plus (Life Technologies), GenePORTER (Gene therapy Systems; San Diego, Calif.) and the Perfect Lipids Transfection Kit (Invitrogen, Carlsbad, Calif.). Lipofectamine Plus was most suitable in our hands and used for all subsequent transfections. Although stable transfectants are preferable to transiently transfected cells, we have been unsuccessful in generating stably transfected ZEM2S cells.

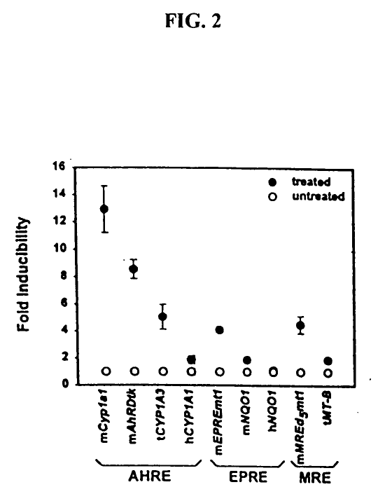

Comparing the potency of various mammalian and trout promoters for their capacity to confer dose-dependent LUC induction, we examined four AHRE, three EPRE and two MRE constructs (FIG. 2). All nine promoters that we tested demonstrated dose-dependent LUC induction upon treatment with the appropriate environmental agent (not shown). For the prototypic inducers of the three classes of environmental inducers, we decided to use dioxin, tBHQ and cadmium, respectively (FIG. 2). From the magnitude of successful responses in the ZEM2S cell line, we chose the AHRDtk, EPREmt1 and MREd5mt1 constructs as the best three candidates for developing transgenic zebrafish.

Although the present invention has been discussed with respect to the preferred and alternative embodiments, it will be apparent to those skilled in the art that the present invention is not limited to these embodiments. Therefore, a person of ordinary skill in the art will understand that variations and modifications of the present invention are within the spirit and scope of the present invention.

What is claimed:

1. A method of measuring contaminants in water comprising:
a. introducing into an aquatic organism a DNA construct comprising a sequence encoding at least one regulatory response element operatively linked to a DNA molecule comprising at least one reporter gene such that the at least one regulatory response element controls the expression of the at least one reporter gene and thereby forming an operative transgenic organism;
b. exposing the transgenic organism to a water sample to be tested for a time sufficient to allow contaminants to become bioconcentrated within the transgenic organism;
c. exposing the transgenic organism to conditions permitting expression of the at least one reporter gene; and
d. detecting the expression of the at least one reporter gene; and
e. correlating the detected expression of the transgenic organism to a reference standard comprising an aquatic source containing a known contaminant concentration and thereby determining the quantity of contaminants in the water sample.

2. A method of measuring contaminants in water comprising:
a. introducing into an organism a DNA construct comprising a sequence of two or more regulatory response elements operatively linked to a DNA molecule comprising at least one reporter gene such that at least one of the regulatory elements controls expression of the reporter gene and thereby forming an operative transgenic organism;
b. exposing the transgenic organism to a water sample to be tested for a time sufficient to allow contaminants to become bioconcentrated within the transgenic organism;
c. exposing the transgenic organism to conditions permitting expression of the reporter gene; and
d. detecting the expression of the reporter gene; and
e. correlating the detected expression of the transgenic organism to a reference standard comprising an aquatic source containing a known contaminant concentration and thereby determining the quantity of contaminants in the water sample.

3. The method according to claim 1 wherein the regulatory response element is a promoter.

4. The method according to claim 1 wherein the regulatory response element is a promoter selected from the group consisting of metal response elements (MRE), aromatic hydrocarbon response elements (AHRE), estrogen response elements (ERE), electrophile response elements (EPRE), and retinoic acid response elements (RARE, RXRE).

5. The method according to claim 5 wherein the transgenic organism is exposed to a water sample containing a known amount of contaminant.

6. The method according to claim 5 wherein the transgenic organism is exposed to the water sample for at least one minute.

7. The method according to claim 5 wherein the transgenic organism is exposed to the water sample for at least 2 minutes.

8. The method according to claim 5 wherein the transgenic organism is exposed to the water sample for at least one hour.

9. The method according to claim 5 wherein the transgenic organism is exposed to the water sample for at least 12 hours.

10. The method according to claim 5 wherein the transgenic organism is exposed to the water sample for at least 24 hours.

11. The method according to claim 2 wherein the regulatory response element is a promoter selected from the group consisting of metal response elements (MRE), aromatic hydrocarbon response elements (AHRE), estrogen response elements (ERE), electrophile response elements (EPRE), and retinoic acid response elements (RARE, RXRE).

12. The method according to claim 11 wherein the reporter gene encodes a bioluminescent molecule.

13. The method according to claim 4 wherein the DNA construct is made up of multiple copies of the same response element.

14. The method according to claim 4 wherein the DNA construct contains more than one type of response element.

15. The method according to claim 4 wherein the DNA construct contains more than two types of response element.

16. The method according to claim 4 wherein the DNA construct contains two or more copies each of more than one type of response element.

17. The method according to claim 4 wherein the DNA construct contains additional promoters or enhancers.

18. The method according to claim 4 wherein the DNA construct contains additional genes for trascription.

19. The method according to claim 4 wherein the reporter gene encodes a bioluminescent molecule.

20. The method according to claim 19 wherein the reporter gene is a luciferase or GFP gene.

21. The method according to claim 19 wherein the reporter gene is a luciferase gene.

22. The method according to claim 19 wherein the reporter gene is a eucaryotic luciferase gene.

23. The method according to claim 19 wherein the reporter gene is a GFP reporter gene.

24. The method according to claim 22 wherein the conditions permitting expression of the reporter gene include a sufficient amount of enzyme substrate.

25. The method according to claim 24 wherein the substrate is luciferin.

26. The method according to claim 25 wherein the detection of the expression of the reporter gene is by using a luminometer.

27. The method according to claim 4 wherein the transgenic organism is exposed to a water sample to be tested continually wherein the organism is removed from the water sample repeatedly at selected intervals exposed to conditions permitting expression of the reporter gene and detected for reporter gene expression wherein such repeated exposures and detecting of expression is effective to track a time course of contaminant levels.

28. The method according to claim 22 wherein the contaminant to be detected is one or more contaminants selected from the group consisting of polyaromatic hydrocarbons, electrophilic oxidants, heavy metals, endocrines, and retinoids.

29. The method according to claim 22 wherein the contaminant to be detected is one or more contaminants selected from the group consisting of 2,3,7,8-tetrachlorodibenzo-p-dioxin, dioxin, polychlorinated biphenyls, quinones, mercury, copper, nickel, cadmium, zinc, estrogens, retinoic acid and 9-cis-retinoic acid.

30. The method according to claim 22 wherein the contaminant to be detected is mercury.

31. The method according to claim 28 wherein both a polyaromatic hydrocarbon and an electrophilic oxidant heavy metal are detected contaminants.

32. The method according to claim 22 wherein the contaminants become bioconcentrated at least 1,000-fold, relative to the water in the tissues of the organism.

33. The method according to claim 22 wherein the fish are removed from the test water and placed immediately in a luminometer cuvette and incubated with luciferin.

34. The method according to claim 4 wherein the reporter gene sequence has a degree of homology of at least about 85% to the reporter gene sequence of the native source of the reporter gene.

35. The method according to claim 22 wherein the reporter gene has at least 85% homology to a luciferase reporter gene in the firefly Photinus pyralis.

36. The method according to claim 23 wherein the reporter gene sequence has at least 85% homology to a reporter gene sequence of a species of Aequorea.

37. The method according to claim 22 wherein the luciferase reporter gene is derived from a species selected from the group consisting of Aequorea victoria and Aequorea forskalea.


SEQUENCE LISTING (6 sequences)

SEQ ID NO: 1 - DNA, 7 bp, Artificial Sequence
  Response element AHRE
twgcgtg

SEQ ID NO: 2 - DNA, 10 bp, Artificial Sequence
  misc_feature (1)...(1): n=a,t,g, or c; r=a or g; w=a or t
rtgacnnngc

SEQ ID NO: 3 - DNA, 9 bp, Artificial Sequence
  misc_feature (4)...(4): n=a,t,g, or c; r=a or g; w=a or t
tgcrcncgg

SEQ ID NO: 4 - DNA, 13 bp, Artificial Sequence
  misc_feature (6)...(8): n=a,t,g, or c; r=a or g; w=a or t
ggtcannntgacc

SEQ ID NO: 5 - DNA, 13 bp, Artificial Sequence
  misc_feature (1)...(1): n=a,t,g, or c; r=a or g; w=a or t
rggtcanrggtca

SEQ ID NO: 6 - DNA, 29 bp, Artificial Sequence
  Response element RXRE
ggggtcaaaggtcaggggtcatggggtca